(19) United States
(12) Patent Application Publication
(10) Pub. No.: US 2021/0208136 A1
(43) Pub. Date: Jul. 8, 2021

(54) ULTRASENSITIVE METHOD FOR MULTIPLEXED DETECTION OF BIOMARKERS

(71) Applicant: SEOUL NATIONAL UNIVERSITY R&DB FOUNDATION, Seoul (KR)

(72) Inventors: Sungchul HOHNG, Seoul (KR); Jongjin LEE, Cheongju-Si (KR)

(21) Appl. No.: 16/975,875

(22) PCT Filed: Mar. 12, 2019
(86) PCT No.: PCT/KR2019/002857

§ 371 Date: Aug. 26, 2020

(30) Foreign Application Priority Data
Mar. 13, 2018 (KR) 10-2018-0029473

(51) Int. Cl. G01N 33/543; C12Q 1/6834; C12Q 1/6818; G01N 21/64

(52) U.S. Cl. CPC G01N 33/54306; C12Q 1/6834; G01N 2021/6439; G01N 21/6428; C12Q 1/6818

(57) ABSTRACT

The present invention relates to an ultrasensitive method for multiplexed detection of biomarkers. More specifically, the present invention provides a method for multiplexed detection of biomarkers, including a) attaching one or more types of biomarkers present in a sample taken from a subject to the surface of a substrate, b) attaching docking strands to the biomarkers and allowing detection antibodies to specifically bind to the biomarkers, and binding the detection antibodies to the corresponding biomarkers, c) binding imager strands labeled with fluorescent molecules or combinations of donor and acceptor strands to the docking strands to generate a fluorescence signal, and d) detecting the fluorescence signal. Steps c) and d) are repeated as many times as the number of the biomarker types by removing the used imager strands or the used donor and acceptor strands and introducing new imager strands or different combinations of donor and acceptor strands to the docking strands.

### TECHNICAL FIELD

This application claims priority to Korean Patent Application No. 10-2018-0029473 filed on Mar. 13, 2018, the disclosure of which is incorporated herein by reference in its entirety.

The present invention relates to an ultrasensitive method for multiplexed detection of biomarkers. More specifically, the present invention relates to a method for multiplexed detection of biomarkers, including a) attaching one or more types of biomarkers present in a sample taken from a subject to the surface of a substrate, b) attaching docking strands to the detection antibodies and allowing detection antibodies to specifically bind to the corresponding biomarkers, c) binding imager strands labeled with fluorescent molecules or combinations of donor and acceptor strands to the docking strands to generate a fluorescence signal, and d) detecting the fluorescence signal, wherein steps c) and d) are repeated as many times as the number of the biomarker types by removing the used imager strands or the used donor and acceptor strands and introducing new imager strands or different combinations of donor and acceptor strands to the docking strands, and an ultrasensitive kit for multiplexed detection of biomarkers using the method.

### BACKGROUND ART

Changes in the level of biomarkers for diseases are different from person to person, which explains the necessity of databases for individuals. Frequent diagnostic examinations are required for the construction of databases for individuals and early diagnosis of diseases. To this end, the use of biofluids (for example, blood) necessary for diagnosis needs to be minimized and the diagnostic costs should be lowered. Since the levels of several biomarkers for one disease vary simultaneously or the level of one biomarker varies by several diseases, the level of only one biomarker cannot be used for an accurate diagnosis. That is, accurate diagnosis of various diseases requires simultaneous measurement of different types of biomarkers. Consequently, multiplexed diagnostic functions to enable simultaneous analysis of various biomarkers with high sensitivity are essential for early diagnosis of diseases using biomarkers.

ELISA-based techniques have mainly been used to detect biomarkers and require at least several tens of microliters of blood because they have a sensitivity in the range of several to several tens of pg/ml. Since ELISA-based techniques cannot be used for multiplexed detection, the detection of a number of biomarkers requires a sufficient amount of blood and a sufficient number of detection kits in proportion to the number of the biomarkers. The sampling of a large amount of blood imposes mental and physical burdens on subjects and the use of a large number of detection kits creates an economic burden. For these reasons, the use of biomarkers for the early diagnosis of diseases is very limited. Currently, biomarkers are used for monitoring the prognosis of specific diseases.

The development of single-molecule fluorescence microscopy allows the observation of individual molecules. When applied to biomarker detection, single-molecule fluorescence microscopy has a sensitivity in the fg/ml range, which is approximately 1000 times higher than that of existing fluorescence imaging techniques (Nature 473, 484-488, 2011). According to a general fluorescence imaging technique, fluorescent molecules are immobilized onto molecules to be observed such that different types of biomarkers are distinguished using fluorescence signals. Although the different types of biomarkers are distinguished based on the different colors of the fluorescent molecules, only 3-4 types of the fluorescent molecules can be distinguished and observed with general image sensors. Consequently, the use of single-molecule fluorescence microscopy, whose sensitivity is in the fg/ml range corresponding to approximately 1000 times that of existing techniques, enables the detection of only 3-4 types of biomarkers.

The application of DNA-PAINT technology (Nature Methods 11, 313-318, 2014) to single-molecule fluorescence microscopy allows simultaneous analysis of a variety of biomarkers while maintaining the high sensitivity.

According to this convergence technology, detection antibodies conjugated with docking strands other than fluorescent molecules are allowed to bind to analyte biomarkers, fluorescent molecules are labeled on imager strands that are attached to the docking strands for a while (several seconds) and detached from the docking strands, and a fluorescence signal generated when the imager strands bind to the docking strands is detected. In this way, many types of biomarkers can be detected with single-color fluorescent molecules while maintaining high sensitivity.

However, when DNA-PAINT technology is used, high background noise is generated because all imager strands emit fluorescence signals irrespective of whether they are bound to docking strands or not. Thus, the concentration of the imager strands is limited to several nM to several tens of nM, disadvantageously resulting in a low detection rate.

Under these circumstances, there is a need to develop methods for rapid analysis of a variety of biomarkers while maintaining the high sensitivity of single-molecule fluorescence microscopy.

### DETAILED DESCRIPTION OF THE INVENTION

### Problems to be Solved by the Invention

The present inventors have made an effort to find a method for rapid analysis of various biomarkers while maintaining high sensitivity, and as a result, found that the application of FRET-PAINT technology to single-molecule fluorescence microscopy allows for more rapid detection of biomarkers. The present invention has been accomplished based on this finding.

Therefore, one object of the present invention is to provide a method for multiplexed detection of biomarkers, including a) attaching one or more types of biomarkers present in a sample taken from a subject to the surface of a substrate, b) allowing docking strand-conjugated detection antibodies to specifically bind to the biomarkers, c) binding imager strands labeled with fluorescent molecules or combinations of donor and acceptor strands to the docking strands to generate a fluorescence signal, and d) detecting the fluorescence signal, wherein steps c) and d) are repeated as many times as the number of the biomarker types by removing the used imager strands or the used donor and acceptor strands and introducing new imager strands or new combinations of donor and acceptor strands to the docking strands

A further object of the present invention is to provide a kit for multiplexed detection of biomarkers, including

a) one or more types of detection antibodies conjugated with docking strands and

b) i) one or more types of imager strands labeled with fluorescent molecules or

ii) one or more types of donor strands labeled with fluorescent molecules and one or more types of acceptor strands labeled with fluorescent molecules

wherein the kit detects a signal generated when the detection antibodies conjugated with the docking strands bind to biomarkers and the imager strands or the donor and acceptor strands bind to the docking strands.

Another object of the present invention is to provide a method for multiplexed detection of biomarkers, including a) attaching one or more types of RNAs present in a sample taken from a subject to the surface of a substrate, b) binding imager strands labeled with fluorescent molecules or combinations of donor and acceptor strands to the RNAs to generate a fluorescence signal, and c) detecting the fluorescence signal, wherein steps b) and c) are repeated as many times as the number of the RNA types by removing the used imager strands or the used donor and acceptor strands and introducing new imager strands or different combinations of donor and acceptor strands to the RNAs.

Still another object of the present invention is to provide a kit for multiplexed detection of biomarkers, including

a) i) one or more types of imager strands labeled with fluorescent molecules; or

ii) one or more types of donor strands labeled with fluorescent molecules and one or more types of acceptor strands labeled with fluorescent molecules;

wherein the kit detects a signal generated when the imager strands or the donor and acceptor strands bind to RNAs.

### Means for Solving the Problems

One aspect of the present invention provides a method for multiplexed detection of biomarkers, including a) attaching one or more types of biomarkers present in a sample taken from a subject to the surface of a substrate, b) allowing docking strand-conjugated detection antibodies to specifically bind to the biomarkers, c) binding imager strands labeled with fluorescent molecules or combinations of donor and acceptor strands to the docking strands to generate a fluorescence signal, and d) detecting the fluorescence signal, wherein steps c) and d) are repeated as many times as the number of the biomarker types by removing the used imager strands or the used donor and acceptor strands and introducing new imager strands or new combinations of donor and acceptor strands to the docking strands.

A further object of the present invention is to provide a kit for multiplexed detection of biomarkers, including

a) one or more types of detection antibodies conjugated with docking strands and

b) i) one or more types of imager strands labeled with fluorescent molecules or

ii) one or more types of donor strands labeled with fluorescent molecules and one or more types of acceptor strands labeled with fluorescent molecules

wherein the kit detects a signal generated when the detection antibodies conjugated with the docking strands bind to biomarkers and the imager strands or the donor and acceptor strands bind to the docking strands.

Another aspect of the present invention provides a method for multiplexed detection of biomarkers, including a) attaching one or more types of RNAs present in a sample taken from a subject to the surface of a substrate, b) binding imager strands labeled with fluorescent molecules or combinations of donor and acceptor strands to the RNAs to generate a fluorescence signal, and c) detecting the fluorescence signal, wherein steps b) and c) are repeated as many times as the number of the RNA types by removing the used imager strands or the used donor and acceptor strands and introducing new imager strands or different combinations of donor and acceptor strands to the RNAs.

Yet another aspect of the present invention provides a kit for multiplexed detection of biomarkers, including

a) i) one or more types of imager strands labeled with fluorescent molecules; or

ii) one or more types of donor strands labeled with fluorescent molecules and one or more types of acceptor strands labeled with fluorescent molecules;

wherein the kit detects a signal generated when the imager strands or the donor and acceptor strands bind to RNAs.

The present invention will now be described in detail.

The present invention provides a method for multiplexed detection of biomarkers, including a) attaching one or more types of biomarkers present in a sample taken from a subject to the surface of a substrate, b) allowing docking strand-conjugated detection antibodies to specifically bind to the biomarkers, c) binding imager strands labeled with fluorescent molecules or combinations of donor and acceptor strands to the docking strands to generate a fluorescence signal, and d) detecting the fluorescence signal, wherein steps c) and d) are repeated as many times as the number of the biomarker types by removing the used imager strands or the used donor and acceptor strands and introducing new imager strands or new combinations of donor and acceptor strands to the docking strands.

A description will be given of the individual steps of the method according to the present invention.

In step a), one or more types of biomarkers present in a sample taken from a subject are attached to the surface of a substrate. Preferably, one or a plurality of types of biomarkers are attached to the surface of a substrate.

As used herein, the term “subject” refers to an animal who is to be diagnosed for various diseases. The subject is preferably a mammal, particularly animal including a human, more preferably a patient in need of treatment.

The sample is isolated from a subject suspected of having a disease and can be selected from the group consisting of, but not limited to, tissue, blood, serum, plasma, saliva, mucus, and urine. Most preferably, the sample is blood, plasma, serum, saliva, tissue fluid or urine.

The sample may be diluted with a biomarker-free solution. The concentrations of the biomarkers in the sample may greatly vary from fg/ml to mg/ml depending on the biomarker types. Preferably, the sample is diluted in various ratios such that proper numbers of the biomarker molecules are detected by an image sensor. The same sample is preferably diluted in different ratios, most preferably in weight ratios of 1:10 to 1000.

In step b), allowing docking strand-conjugated detection antibodies to specifically bind to the biomarkers.

The term “antibodies” is used in its broadest sense to encompass capture antibodies and detection antibodies. Specifically, the antibodies include monoclonal antibodies (including full-length monoclonal antibodies), polyclonal antibodies, multispecific antibodies (e.g., bispecific antibodies), and antibody fragments (e.g., variable regions and other regions of antibodies that exhibit desired bioactivities). The antibodies are intended to include both monoclonal and polyclonal antibodies, and also include chimeric antibodies, humanized antibodies, and human antibodies.

Preferably, the antibodies include Fab, F(ab)2, Fab′, F(ab′)2, Fv, diabodies, nanobodies, or scFv. More preferably, the antibodies are scFv, Fab, nanobodies and immunoglobulin molecules.

The docking strands can be conjugated to the detection antibodies by general methods known in the art for binding protein molecules to nucleic acid molecules, for example, based on a binding reaction between nucleic acid molecules and protein molecules using a compound having two different reactive groups, for example, succinimidyl 4-(N-maleimidomethyl)cyclohexane-1-carboxylate (SMCC) in which the NHS-ester group reacts with an amine and the maleimide group reacts with a thiol. The docking strands can be conjugated to the detection antibodies by reacting the thiol group-containing docking strands with SMCC and reacting the resulting products with the amine groups at the N-termini of the detection antibodies or the amine groups of lysine. The docking strands can also be conjugated to the detection antibodies using a commercially available kit.

For accurate measurement, the detection antibodies are primary antibodies and the docking strands are conjugated to the primary antibodies. For rapid measurement, the detection antibodies are a combination of primary antibodies and secondary antibodies and the docking strands are conjugated to the secondary antibodies.

The primary antibodies refer to immunoglobulin molecules that specifically bind to biomarkers and their type is not limited. In the Examples section that follows, anti-tubulin antibodies and anti-Tom20 antibodies were used as the primary antibodies.

The secondary antibodies refer to antibodies that bind to the primary antibodies bound to biomarkers and their type is not limited. In the Examples section that follows, donkey anti-rabbit IgG antibodies or donkey anti-rat IgG antibodies were used as the secondary antibodies.

In step c), imager strands labeled with fluorescent molecules or combinations of donor and acceptor strands are allowed to bind to the docking strands to generate a fluorescence signal.

The docking strands, the donor strands, and the acceptor strands are individually selected from the group consisting of nucleic acids and nucleic acid analogs (DNA, RNA, PNA or LNA) single strands capable of complementary binding. The docking strands are conjugated to the detection antibodies and the imager strands, the donor strands, and the acceptor strands complementarily bind to the docking strands. The imager strands are used when the DNA-PAINT technique is employed. The donor strands and the acceptor strands are used when the FRET-PAINT technique is employed.

The imager strands have different sequences depending on the biomarker types. For example, in the case where two imager strands bind to two different types of biomarkers, their sequences are different in at least one base, indicating that the sequences of the imager strands bound to the different types of biomarkers are different irrespective of the number of the imager strands.

According to one embodiment of the present invention, the donor strands and the acceptor strands have different sequences depending on the biomarker types. For example, the use of N donor strands and N acceptor strands having different sequences is necessary for detecting N different types of biomarkers. That is, the sequences of the donor strands are different in at least one base, the sequences of the acceptor strands binding to the different types of biomarkers are also different in at least one base, and all donor and acceptor strands have different sequences.

According to one embodiment of the present invention, the acceptor strands may have the same sequence as some or all of the biomarkers; and the sequences of the donor strands may be different from those of the acceptor strands and may be different from each other depending on the biomarker types. For example, when it intends to detect two different types of biomarkers, the sequences of donor strands binding to the different biomarkers may be different in at least one base and acceptor strands having the same sequence may be used for detection of the biomarkers.

According to one embodiment of the present invention, the donor strands may have the same sequence as some or all of the biomarkers; and the sequences of the acceptor strands may be different from those of the donor strands and may be different each other depending on the biomarker types. For example, when it intends to detect two different types of biomarkers, the sequences of acceptor strands binding to the different biomarkers may be different in at least one base and donor strands having the same sequence may be used for detection of the biomarkers.

The docking strands include sequences complementary to the sequences of the imager strands. The docking strands include sequences complementary to the sequences of both the donor strands and the acceptor strands.

The term “fluorescent molecules” as used herein refers to molecules of a detectable fluorescent dye compound that is labeled on the imager strands, the donor strands, and the acceptor strands. Examples of the fluorescent molecules include, but are not limited to, Rhodamine fluorescent molecules, Alexa fluorescent molecules, fluorescein isothiocyanate (FITC) fluorescent molecules, 5-carboxyfluorescein (FAM) fluorescent molecules, Atto fluorescent molecules, BODIPY fluorescent molecules, CF fluorescent molecules, Cy fluorescent molecules, DyLight Fluor fluorescent molecules, Texas Red fluorescent molecules, and fluorescein fluorescent molecules. Most preferably, the fluorescent molecules are Alexa Fluor, Atto, BODIPY, CF, Cy or DyLight Fluor fluorescent molecules.

The peak wavelength of the emission spectrum of the fluorescent molecules labeled on the donor strands is shorter than the peak wavelength of the absorption spectrum of the fluorescent molecules labeled on the acceptor strands.

In step d), the fluorescence signal is detected. Steps c) and d) are repeated as many times as the number of the biomarker types by removing the used imager strands or the used donor and acceptor strands and introducing new imager strands or new combinations of donor and acceptor strands to the docking strands.

The fluorescence signal is generated when the imager strands bind to the docking strands or combinations of the donor and acceptor strands bind to the docking strands. Specifically, the fluorescence signal may be generated when the imager strands remain bound to the docking strands or the fluorescent molecules (donors) labeled on the donor strands are located very close to the fluorescent molecules (acceptors) labeled on the acceptor strands by the docking strands. The fluorescence signal can be detected using a highly sensitive image sensor (e.g., EMCCD, sCMOS or iCMOS).

The term “biomarkers” as used herein refers to biomaterials that can predict disease, health, and physiological states. Biomarkers are used in many fields of science. Biomarkers are also used to measure or evaluate biological treatment processes, pathogenetic processes, and pharmacological processes for therapeutic applications. For example, biomarkers are used as materials that determine specific stages of diseases to recognize the presence of antibodies against infection. Biomarkers are used to determine the state and variation of proteins associated with the progress of diseases and the sensitivity of the disease to given treatment regimens.

The biochemical biomarkers may be polypeptides, peptides, nucleic acids, proteins or metabolites that can be found in body fluids such as blood, saliva, and urine. Examples of such biochemical biomarkers include, but are not limited to, interleukins, CD24, CD40, integrin, cystatin, interferons, tumor necrosis factor (TNF), MCP, VEGF, GLP, ICA, HLA-DR, ICAM, EGFR. FGF, BRAF, GREB, FRS, LZTS, CCN, mucin, leptin, apolipoproteins, tyrosine, neuron adhesion molecules analogous proteins, fibronectin, glucose, uric acid, carbonic anhydrase, and cholesterol.

The biomarkers can be directly attached to the substrate surface without the need to use capture antibodies for rapid and convenient measurement. Alternatively, the biomarker molecules may be attached to capture antibodies immobilized onto the substrate surface through the antigen-antibody reaction for higher accuracy of measurement results.

In the case where the biomarkers are directly attached to the substrate surface, for example, a coverslip is coated with a bead solution and cells corresponding to the sample are cultured and immobilized onto the substrate surface. Capture antibodies may be attached to the substrate by diluting the capture antibodies with a 0.06 M carbonate or bicarbonate buffer solution at pH 9.5 and bringing the dilution into contact with the substrate at a predetermined temperature for a predetermined time. Then, the substrate is treated with a raw or processed sample. The capture antibodies adsorbed to the substrate form complexes with the biomarkers in the sample. The complexes are washed with a buffer such as Tween 20 or a cleaning agent such as distilled water to remove non-specifically bound antibodies or contaminants.

The method of the present invention is not carried out only once. According to the method of the present invention, the imager strands labeled with the fluorescent molecules bind to the docking strands to generate a fluorescence signal, the fluorescence signal is detected, the bound imager strands are removed, different imager strands are allowed to bind to the docking strands to generate a fluorescence signal, the fluorescence signal is detected, and this procedure is repeated as many times as the number of the biomarker types.

Alternatively, the donor and acceptor strands labeled with the fluorescent molecules bind to the docking strands to generate a fluorescence signal, the fluorescence signal is detected, the bound strands are removed, different combinations of donor and acceptor strands are allowed to bind to the docking strands to generate a fluorescence signal, the fluorescence signal is detected, and this procedure is repeated several times, preferably as many times as the number of the biomarker types.

For example, the donor strand consisting of 9 bases may have 49 (=262,144) sequences. Since the number of the sequences of the donor strands is larger than the number of all proteins in the human body, the donor and acceptor strands having different sequences are assigned to all biomarkers so that the biomarkers can be measured sequentially.

The present invention also provides a kit for multiplexed detection of biomarkers, including a) one or more types of detection antibodies conjugated with docking strands and b) i) one or more types of imager strands labeled with fluorescent molecules or ii) one or more types of donor strands labeled with fluorescent molecules and one or more types of acceptor strands labeled with fluorescent molecules wherein the kit detects a signal generated when the detection antibodies conjugated with the docking strands bind to biomarkers and the imager strands or the donor and acceptor strands bind to the docking strands.

The kit includes i) a substrate, ii) capture antibodies, iii) primary detection antibodies conjugated with docking strands, or primary detection antibodies unconjugated with docking strands and secondary detection antibodies conjugated with docking strands, and iv) imager strands labeled with fluorescent molecules, or donor and acceptor strands. Preferably, the kit consists of a substrate, capture antibodies, primary detection antibodies conjugated with docking strands, donor strands, and acceptor strands.

The docking strands, the imager strands, the donor strands, and the acceptor strands are individually selected from the group consisting of nucleic acids and nucleic acid analogs (DNA, RNA, PNA, or LNA).

Biomarkers present in a sample may be directly attached to the substrate surface. Alternatively, the capture antibodies capable of capturing biomarkers may be attached to the substrate surface. In this case, the substrate surface is previously modified. Alternatively, the capture antibodies may be previously attached to the substrate surface. The substrate may be selected from the group consisting of slide glass, coverslips, quartz, and plastics but is preferably a #1 or #1.5 coverslip.

Preferably, the kit of the present invention uses docking strands, donor strands, and acceptor strands to detect biomarkers. The kit of the present invention may further include a tool and/or a reagent known in the art that is used to detect biomarkers by fluorescence microscopy or ELISA. The kit of the present invention may further include a tube where the components are mixed, a well plate, and an instruction manual on how to use it.

The kit of the present invention may be a research use only (RUO) kit, an investigational use only (IUO) kit or an in vitro diagnostic (IVD) kit. The IVD kit is intended to include in vitro companion diagnostics (IVD-CDx) kits.

The present invention also provides a method for multiplexed detection of biomarkers, including a) attaching one or more types of RNAs present in a sample taken from a subject to the surface of a substrate, b) binding imager strands labeled with fluorescent molecules or combinations of donor and acceptor strands to the RNAs to generate a fluorescence signal, and c) detecting the fluorescence signal, wherein steps b) and c) are repeated as many times as the number of the RNA types by removing the used imager strands or the used donor and acceptor strands and introducing new imager strands or different combinations of donor and acceptor strands to the RNAs.

A description will be given of the individual steps of the method according to the present invention.

In step a), one or more types of RNAs present in a sample taken from a subject are attached to the surface of a substrate.

The ribonucleic acids (RNAs) may function as biomarkers to be detected. The RNAs are preferably non-coding RNAs. Non-coding RNAs are types of RNAs that lack amino acid codes for specific proteins. Unlike mRNAs that translate gene sequences, non-coding RNAs exist in various forms and can be produced from mRNA by-products or degradation products or through separate transcription processes. Non-coding RNAs are known to play a role in the regulation of chromosomal activity or protein expression. The analyte RNAs are intended to include small RNAs such as miRNAs, snRNAs, snoRNAs, aRNAs, siRNAs, piRNAs, exRNAs, and scaRNAs, but are preferably microRNAs.

microRNAs (miRNAs) are small non-coding RNA molecules consisting of approximately 22 bases. miRNAs are involved in the regulation of gene expression after RNA silencing and transcription. miRNAs are found in numerous biofluids such as blood and urine and their levels vary by various diseases. For these reasons, miRNAs are receiving attention as biomarkers.

RNAs cannot be captured by the antigen-antibody reaction. In the present invention, the analyte RNAs are attached to the substrate surface by immobilizing capture probes having sequences complementary to portions of the analyte RNAs onto a substrate and allowing the RNAs to complementarily bind to the capture probes.

The sample may be diluted with an RNA-free solution. The concentrations of the RNAs in the sample may greatly vary depending on the RNA types. Accordingly, the sample can be diluted in various ratios such that appropriate numbers of the RNA molecules are detected by an image sensor.

In step b), imager strands labeled with fluorescent molecules or combinations of donor and acceptor strands are allowed to bind to the RNAs to generate a fluorescence signal.

In the present invention, the imager strands or the donor and acceptor strands are sequenced such that they can complementarily bind to single-stranded portions of the RNAs which are not complementarily bound to the capture probes.

The capture strands, the imager strands, the donor strands, and the acceptor strands are individually selected from the group consisting of nucleic acids and nucleic acid analogs (DNA, RNA, PNA, or LNA) strands.

In step c), the fluorescence signal is detected. Steps b) and c) are repeated as many times as the number of the RNA types by removing the used strands.

The fluorescence signal is generated when the imager strands or both the donor and acceptor strands bind to single-stranded portions of the RNAs which are not complementarily bound to the capture probes. More specifically, the fluorescence signal may be generated when the imager strands remain bound to single-stranded portions of the RNAs which are not complementarily bound to the capture or the fluorescent molecules (donors) labeled on the donor strands are located very close to the fluorescent molecules (acceptors) labeled on the acceptor strands by the RNAs. The fluorescence signal can be detected using a highly sensitive image sensor (e.g., EMCCD, sCMOS or iCMOS).

The present invention also provides a kit for multiplexed detection of biomarkers, including a) i) one or more types of imager strands labeled with fluorescent molecules or ii) one or more types of donor strands labeled with fluorescent molecules and one or more types of acceptor strands labeled with fluorescent molecules wherein the kit detects a signal generated when the imager strands or the donor and acceptor strands bind to RNAs.

The kit includes i) a substrate, ii) capture probes, iii) fluorescent molecule-labeled imager strands, or donor strands, and acceptor strands. Preferably, the kit consists of a substrate, capture probes, donor strands, and acceptor strands.

The capture probes may be previously attached to the substrate. Alternatively, the substrate may be surface modified such that the capture probes can be attached to the substrate surface. The substrate may be selected from the group consisting of slide glass, coverslips, quartz, and plastics but is preferably a #1 or #1.5 coverslip.

In the Examples section that follows, a determination was made as to whether FRET-PAINT can be performed by microscopic examination. To this end, docking strands were immobilized on the surface of the quartz, donor and acceptor strands labeled with fluorescent molecules were injected, and single-molecule images were then recorded, with the result that clear fluorescence intensities were found depending on the concentrations of the donor and acceptor strands (see Example 1 and FIG. 1c).

In the Examples section that follows, the same experiment was conducted using two FRET pairs (Cy3-Cy5 and Alexa488-Cy5). As a result, the Alexa488-Cy5 FRET pair gave the highest fluorescence signal when the gap between donor and acceptor strands was 2 nt, whereas the Cy3-Cy5 FRET pair gave the highest fluorescence signal when the gap between donor and acceptor strands was 6 nt (see Example 1 and FIG. 1d).

In the Examples section that follows, super-resolution fluorescence images were measured and the signal-to-noise ratios (SNRs) of DNA-PAINT and FRET-PAINT at varying DNA concentrations were compared. As a result, noise occurred in the single-molecule images when the DNA concentration was >5 nM for DNA-PAINT and when the DNA concentration was >100 nM for FRET-PAINT, demonstrating that the same SNR can be obtained at higher imager concentrations for FRET-PAINT compared to for DNA-PAINT (see Example 1 and FIGS. 1e to 1k).

In the Examples section that follows, cells were immunostained with anti-tubulin antibody conjugated with docking strands, microtubules were observed using DNA-PAINT, and microtubules in the same area were imaged using FRET-PAINT. The imaging speeds of DNA-PAINT and FRET-PAINT were quantified and compared, with the result that images were recorded at a rate of 10 Hz for a total imaging time of 30 minutes for DNA-PAINT and images were recorded at a rate of 10 Hz for a total imaging time of 60 seconds for FRET-PAINT, demonstrating the imaging speed of FRET-PAINT was found to be higher than that of DNA-PAINT (see Example 2 and FIGS. 2a to 2e).

In the Examples section that follows, cells were immunostained with anti-tubulin antibody and anti-Tom20 antibody conjugated with docking strands, treated with donor and acceptor strands sequentially or simultaneously, and images were recorded, with the result that the sequential treatment with the donor and acceptor strands was found to be more effective in multiplexed imaging (see Example 3 and FIGS. 3a to 3h).

In the Examples section that follows, the numbers of molecules found in images were measured at varying biomarker concentrations. As a result, the individual molecules were well distinguished in the range of 10 pg/mg to 400 pg/ml, enabling the measurement of the biomarker concentrations. In contrast, the molecules started to overlap at concentrations above 1000 pg/ml, making it impossible to measure accurate concentrations. After the sample was diluted in an appropriate ratio such that the concentration reached approximately 100 pg/ml, the number of the molecules was measured and divided by the dilution ratio to calculate the biomarker concentration, which exactly coincides with the original one. An accurate measurement was possible in the concentration range of currently widely used biomarkers (several pg/ml to several tens of ng/ml) (see Example 4 and FIGS. 4a to 4c).

### Effects of the Invention

The ultrasensitive method for multiplexed detection of biomarkers according to the present invention uses a single-molecule fluorescence microscope and a DNA-PAINT or FRET-PAINT system. The method of the present invention enables the detection of various biomarkers from a very small amount of biofluid with a 1000-fold higher sensitivity than conventional methods. Therefore, the method of the present invention is useful for the early diagnosis of various diseases, including cancer, while solving the problems of conventional diagnostic methods using biomarkers.

### BRIEF DESCRIPTION OF THE DRAWINGS

FIG. 1a, FIG. 1b, FIG. 1c, FIG. 1d, FIG. 1e, FIG. 1f, FIG. 1g, FIG. 1h, FIG. 1i, FIG. 1j and FIG. 1k show the principle and characterization of FRET-PAINT. Specifically, FIG. 1a shows docking strands (SEQ ID NO: 1), donor strands (SEQ ID NO: 2), and acceptor strands (SEQ ID NO: 3) used to characterize FRET-PAINT, FIG. 1b is a schematic diagram of FRET-PAINT, FIG. 1i shows representative Cy5 fluorescence intensity time traces (1000 nM Donor_P1_Alexa488 and 100 nM Acceptor_P11_Cy5), FIG. 1d shows normalized FRET efficiency as a function of the donor-acceptor distance for Cy3-Cy5 (empty square) and Alexa488-Cy5 (filled diamond) pairs, FIG. 1e shows DNA-PAINT images of surface-immobilized docking strand (Docking_P0) at indicated concentrations of Acceptor_P11_Cy3, FIG. 1f shows FRET-PAINT images of Docking_P0 at indicated concentrations of Donor_P1_Cy3 with Acceptor_P6_Cy5 fixed at 10 nM, FIG. 1g shows FRET-PAINT images of Docking_P0 at indicated concentrations of Acceptor_P6_Cy5 with Donor_P1_Cy3 fixed at 10 nM, FIG. 1h shows FRET-PAINT images of Docking_P0 at the indicated concentration of Donor_P1_Alexa488 with Acceptor_P2_Cy5 fixed at 10 nM, FIG. 1i shows FRET-PAINT images of Docking_P0 at the indicated concentration of Acceptor_P2_Cy5 with Donor_P1_Alexa488 fixed at 10 nM (Scale bar: 1 m), FIG. 1j compares SNRs of DNA-PAINT at varying Cy3 imager strand concentration (solid line) and FRET-PAINT at varying Cy3 donor strand (bold dashed line), and Cy5 acceptor strand (dotted line) concentrations, and FIG. 1k compares SNRs of DNA-PAINT at varying Cy3 imager strand concentration (solid line) and FRET-PAINT at varying Alexa488 donor strand (bold dashed line), and Cy5 acceptor strand (dotted line) concentrations.

FIG. 2a, FIG. 2b, FIG. 2c, FIG. 2d and FIG. 2e compare the imaging speeds of DNA-PAINT and FRET-PAINT. Specifically, FIG. 2a shows DNA-PAINT images reconstructed at specified acquisition time, FIG. 2b shows FRET-PAINT images of the same area as in FIG. 2a reconstructed at specified acquisition time, FIG. 2c shows the accumulated number of localized spots as a function of time for DNA-PAINT images of FIG. 2a (dashed line), and FRET-PAINT images of FIG. 2b (solid line), FIG. 2d compares the number of localized single-molecule spots per second of FRET-PAINT and DNA-PAINT, and FIG. 2e compares spatial resolutions of DNA-PAINT and FRET-PAINT as a function of imaging time. The error bars represent standard deviation.

FIG. 3a, FIG. 3b, FIG. 3c, FIG. 3d, FIG. 3e, FIG. 3f, FIG. 3g and FIG. 3h show multiplexing capability of FRET-PAINT. Specifically, FIG. 3a schematically shows multiplexed imaging that uses a donor and acceptor strand exchange scheme, FIGS. 3b to 3d show FRET-PAINT images of microtubule (3b) and mitochondria (3c) obtained using the scheme (3a), and (3d) an overlaid image of FIGS. 3b and 3c FIG. 3e schematically shows multiplexed imaging without buffer exchange, and FIGS. 3f to 3h show FRET-PAINT images of microtubule (3f) and mitochondria (3g) obtained using the scheme (3e) and (3h) an overlaid image of FIGS. 3f and 3g (MT, microtubule; MC, mitochondria; DS, donor strand; AS, acceptor strand. Scale bars: 5 m).

FIG. 4a, FIG. 4b and FIG. 4c show the quantification of biomarkers at various concentrations. Specifically, FIG. 4a shows biomarkers at concentrations of 10, 40, 100, 400, 1000, and 10000 pg/ml immobilized in different chambers and measured using donor and acceptor strands, FIG. 4b shows biomarkers at concentrations of 1000 pg/ml and 10000 pg/ml after 1/10 and 1/100 dilution before use, respectively, and FIG. 4c graphically shows the numbers of spots measured at the concentrations indicated in FIGS. 4a and 4b. In FIG. 4c, the 1000 pg/ml data were obtained by counting the number of spots after 1/10 dilution and multiplying the number by 10 and the 10000 pg/ml data were obtained by counting the number of spots after 1/100 dilution and multiplying the number by 100.

### MODE FOR CARRYING OUT THE INVENTION

The present invention will be explained in detail with reference to the following examples. However, these examples are provided for illustrative purposes only and are not intended to limit the scope of the present disclosure.

### Experimental Methods

1. Reagents and Materials

Modified DNA oligonucleotides were purchased from Integrated DNA Technologies. AF488 (Alexa Fluor 488 NHS Ester, catalog number: A20000) was purchased from Thermo Fisher Scientific. Cy3 (Cy3 NHS Ester, catalog number: PA13101) and Cy5 (Cy5 NHS Ester, catalog number: PA15101) were purchased from GE Healthcare Life Sciences. COS-7 cells were purchased from Korean Cell Line Bank. Anti-tubulin antibody (catalog number: ab6160) was purchased from Abcam. Anti-Tom20 antibody (sc-11415) was purchased from Santa Cruz Biotechnology, Inc. Donkey anti-rabbit IgG antibody (catalog number: 711-005-152) and donkey anti-rat IgG antibody (catalog number: 712-005-153) were purchased from Jackson ImmunoResearch Laboratories, Inc. Carboxyl latex beads (catalog number: C37281) were purchased from Thermo Fisher Scientific and paraformaldehyde (catalog number: 1.04005.1000) was purchased from Merck. Glutaraldehyde (catalog number: G5882), Triton X-100 (catalog number: T9284), and Bovine Serum Albumin (BSA, catalog number: A4919) were purchased from Sigma-Aldrich. The docking strands were conjugated to the secondary antibodies using Antibody-Oligonucleotide All-in-One Conjugation Kit (catalog number: A-9202-001) purchased from Solulink.

2. DNA Labeling with Fluorophores

Amine-modified DNA oligonucleotides were labeled with fluorophores which have NHS ester chemical group. 5 μl of 1 mM DNA was mixed with 25 μl of 100 mM sodium tetraborate buffer (pH 8.5). And then 5 μl of 20 mM fluorophore in DMSO was added. The mixture was incubated at 4° C. overnight. 265 μl of distilled water, 900 of ethanol, and 30 of 3 M sodium acetate (pH 5.2) were added and mixed thoroughly. The mixture was incubated at −20° C. for an hour and then centrifuged for a couple of hours. After ethanol was evaporated completely, the DNA pellet was resuspended in 50 of distilled water and the fluorescent labeling efficiency was measured.

3. Cell Culture and Fixation

For drift correction of DNA-PAINT imaging, glass coverslips were coated with carboxyl latex beads.

The coverslip was coated with bead solution 1:10 diluted in distilled water, heated for 10 minutes on a 100° C. hot plate, washed with distilled water, and dried with N₂ gas. COS-7 cells were grown on bead-coated coverslips and then fixed for 10 minutes. 2% glutaraldehyde was used for microtubule imaging and 3% paraformaldehyde and 0.1% glutaraldehyde mixture in PBS buffer was used for microtubule and mitochondria imaging. Fixed samples were stored at 4° C. in PBS buffer until needed.

4. Immunostaining

After cell culture on coverslips and fixation, microtubules were treated with 1:100 diluted anti-tubulin antibodies in a blocking solution (5% BSA and 0.25% Triton X-100 in PBS buffer), incubated at 4° C. overnight, and immunostained. After thorough wash-out of free anti-tubulin antibodies with PBS buffer, cells were incubated with 100 nM secondary antibodies conjugated with docking strands (Docking_P1) for 1 hour. Mitochondria were treated with 1:100 diluted anti-Tom20 antibodies, incubated at 4° C. overnight, and immunostained. After thorough wash-out of free anti-Tom20 antibodies with PBS buffer, cells were incubated with 100 nM secondary antibodies conjugated with docking strands (Docking_P2) for 1 hour at room temperature.

5. Single-Molecule Imaging

For single-molecule imaging, a prism-type total internal reflection fluorescence (TRF) microscopy and highly inclined and laminated optical sheet (HILO) microscopy were used. The microscope was built by modifying a commercial inverted microscope (IX71, Olympus), and equipped with a 100×1.4 NA oil-immersion objective lens (UPanSApo, Olympus).

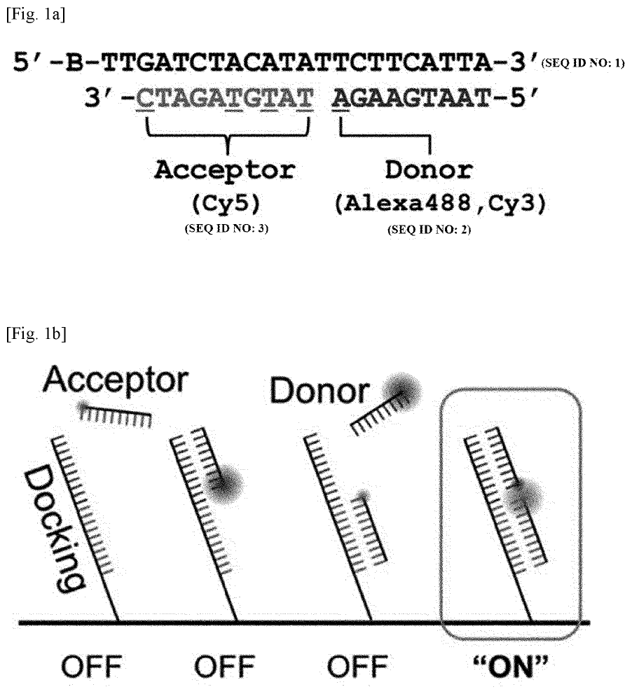

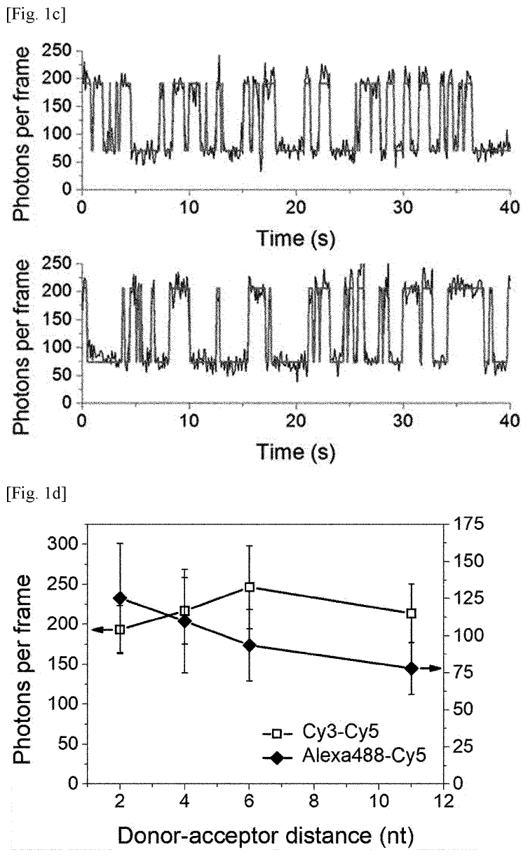

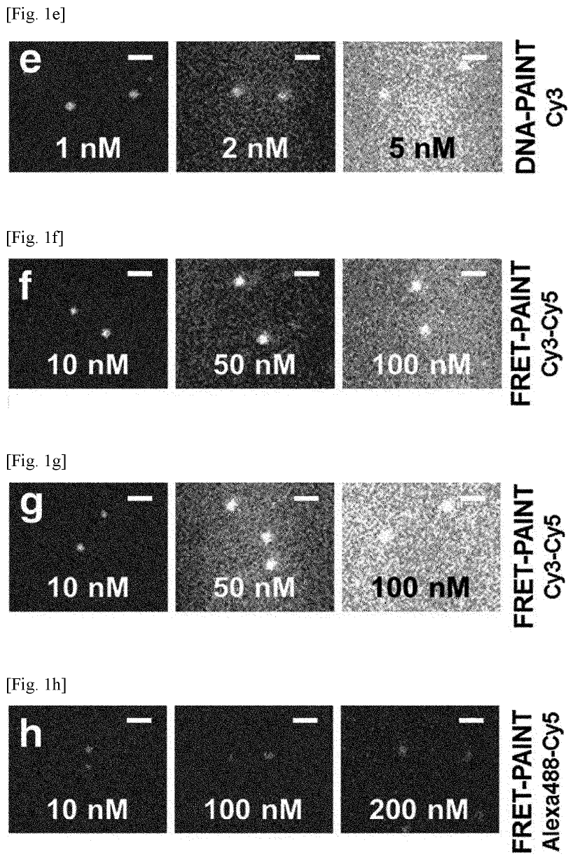

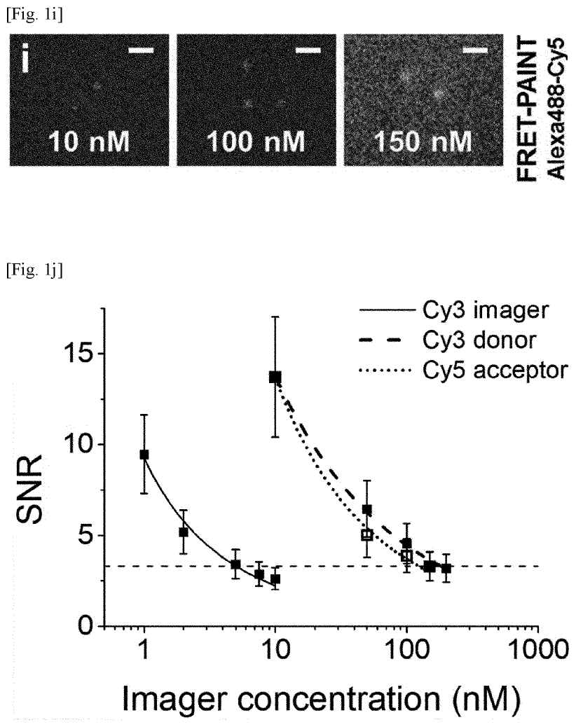

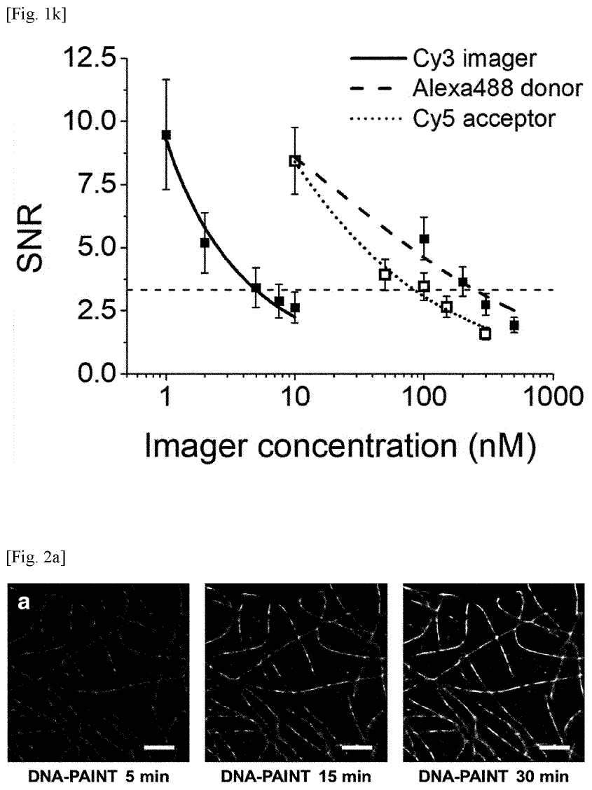

To obtain data in FIG. 1, docking strands were immobilized on the polymer-coated quartz slide surface by using streptavidin-biotin interaction, and donor and acceptor strands were added into the imaging channel. Alexa488, Cy3, and Cy5 were excited by a blue laser (473 nm, 100 mW, MBL-III-473-100 mW, CNI), a green laser (532 nm, 50 mW, Compass 215M-50, Coherent), and a red laser (642 nm, 60 mW, Excelsior-642-60, Spectra-Physics), respectively. Cy3 signal was filtered using a dichroic mirror (640dcxr, Chroma), and Cy5 signal was filtered using a dichroic mirror (740dcxr, Chroma). Single-molecule images were recorded at a frame rate of 10 Hz with electron multiplying charge coupled device (EMCCD) camera (iXon Ultra DU-897UCS0-#BV, Andor).

6. FRET Pair Characterization

To characterize detected photons per frame in FIG. 1d, 13997 (8096), 11021 (5100), 11208 (3451), and 17051 (3980) single-molecule spots were collected for 2 nt, 4 nt, 6 nt, and 11 nt Cy3-Cy5 (Alexa488-Cy5) FRET pairs, respectively. To characterize SNR in FIGS. 1j-1k, 795, 2322, and 742 single-molecule spots were collected for Cy3, Cy3-Cy5 pair, and Alexa488-Cy5 pair, respectively.

7. Drift Correction

For super-resolution imaging with DNA-PAINT, autofocusing and drift correction system based on image correlation method was used. Before filming, one in-focus bright-field image and two out-of-focus images were taken. These three reference images were used to keep track of x, y, and z axes drift. The drift in z-direction was corrected in real-time using a piezo stage (PZ-2000, Applied Scientific Instrumentation) whereas the drift in the xy-plane was corrected during image analysis.

### Example 1: Characterization of FRET-PAINT

Surface-immobilized DNA strands and a TIRF microscope were used to test the feasibility of FRET-PAINT microscopy.

As shown in FIG. 1a, FRET-PAINT used three DNA strands (docking, donor, and acceptor strands). The docking strand (Docking_P0) labeled with a biotin at the 5′-end has two docking sites, each of which base-pairs with the donor or acceptor strand. To increase the FRET probability, a shorter length for the donor strand than for the acceptor strand was chosen whereas relatively longer acceptor strand was used. The donor strand was labeled at the 3′-end with Alexa488 (Donor_P1_Alexa488) whereas the acceptor strand was labeled with Cy5 (Acceptor_P11_Cy5) at the 3′-end.

The sequences of the strands are as follows:

(SEQ ID NO: 1)
Docking_PO = 5′-Biotin-TTGATCTACATATTCTTCATTA-3′
(SEQ ID NO: 2)
Donor_P1_Cy3 - 5′-TAATGAAGA-Cy3-3′
(SEQ ID NO: 2)
Donor_P1_Alexa488 = 5′-TAATGAAGA-Alexa488-3′
(SEQ ID NO: 3)
Acceptor_P2_Cy5 = 5′-Cy5-TATGTAGATC-3′
(SEQ ID NO: 3)
Acceptor_P6_Cy5 = 5′-TATG-Cy5-TAGATC-3′
(SEQ ID NO: 3)
Acceptor_P11_Cy5 = 5′-TATGTAGATC-Cy5-3′

Then, the docking strand was immobilized on the surface of a polymer-coated quartz surface using streptavidin-biotin interaction (FIG. 1b) and took single-molecule images of Cy5 after injecting the donor (1000 nM) and acceptor (100 nM) strands by exciting Alexa488 using a blue laser.

As a result, clear Cy5 fluorescence intensity time traces were obtained at high donor and acceptor concentrations, as shown in FIG. 1c.

The same scheme was carried out to find the optimum labeling position of FRET probes that gives maximum FRET signal for two FRET pairs (Cy3-Cy5 and Alexa488-Cy5). To this end, donor strands labeled with Cy3 (Donor_P1_Cy3) or Alexa488 (Donor_P1_Alexa488) at the 3′-end, and acceptor strands labeled with Cy5 at varying positions (Acceptor_P2_Cy5, P4_Cy5, P6_Cy5, and P11_Cy5).

As a result, the Cy3-Cy5 FRET pair gave the highest Cy5 signal when the gap between donor and acceptor strands was 6 nt, whereas Alexa488-Cy5 FRET pair gave the highest Cy5 signal when the gap between donor and acceptor strands was 2 nt, as shown in FIG. 1d.

Then, super-resolution fluorescence images were recorded using HILO (Highly Inclined and Laminated Optical sheet) microscopy. Signal-to-noise ratios (SNRs) of DNA-PAINT and FRET-PAINT at varying DNA concentrations were compared.

The results are shown in FIGS. 1e to 1i. For DNA-PAINT, noise occurred in the single-molecule images when DNA concentration was above 5 nM (FIG. 1e). For Cy3-Cy5 pair FRET-PAINT, noise occurred when the DNA concentration was above 100 nM (FIGS. 1f and 1g). For Alexa488-Cy5 pair FRET-PAINT, noise occurred when the DNA concentration was above 150 nM (FIGS. 1h and 1i).

These images show that the same SNR can be obtained at higher imager concentrations in FRET-PAINT compared to DNA-PAINT. For instance, 5 nM imager concentration was used for DNA-PAINT to obtain 3.3 SNR, as shown in FIGS. 1j and 1k. For the same SNR, 180 nM donor and 120 nM acceptor concentrations were used for the Cy3-Cy5 pair FRET-PAINT and 250 nM donor and 90 nM acceptor concentrations were used for the Alexa488-C5 pair FRET-PAINT.

### Example 2: Superresolution Imaging with DNA-PAINT and FRET-PAINT

The following experimental procedure was performed to compare the super-resolution imaging speeds of DNA-PAINT and FRET-PAINT.

First, microtubules of COS-7 cells were immunostained with the anti-tubulin antibody which is labeled with Docking_P1.

Then, for DNA-PAINT, microtubules were imaged after injecting 1 nM Cy5-labeled imager strand (Acceptor_P2′_Cy5). For FRET-PAINT, microtubules of the same area were imaged after injecting 30 nM donor (Donor_P1_Alexa488) and 20 nM acceptor (Acceptor_P2_Cy5) strands. Single-molecule images were recorded at a frame rate of 10 Hz, which is fast enough to reliably detect binding of donor and acceptor strands.

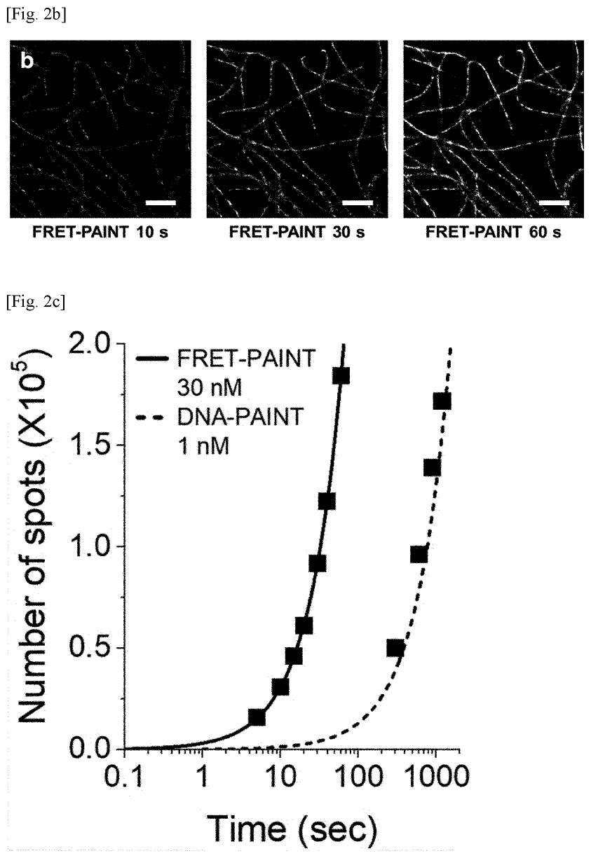

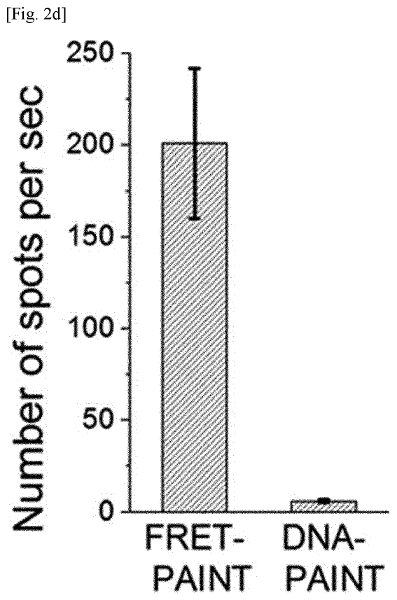

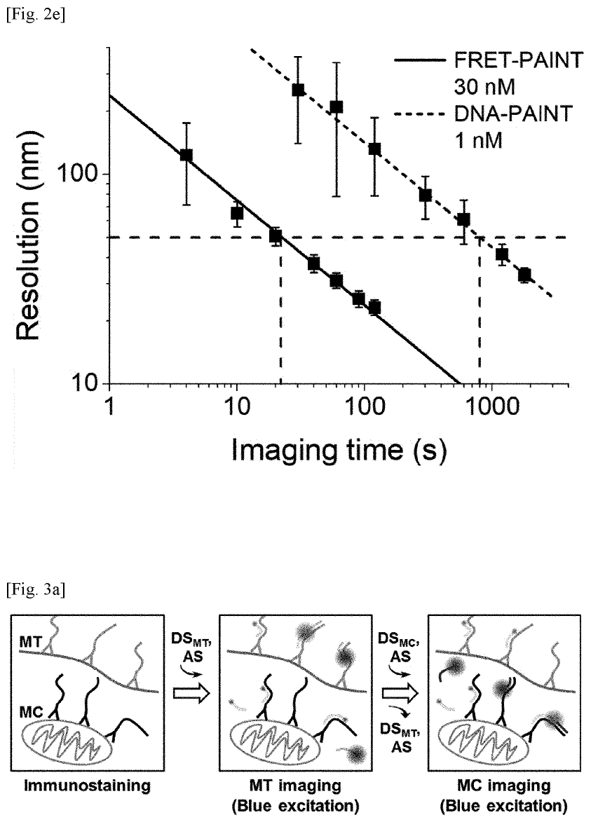

To quantitatively compare the imaging speed of DNA-PAINT and FRET-PAINT, the number of spots of FIGS. 2a and 2b was measured. The same analysis was performed for nine additional imaging areas and the averaged speeds were compared. The convolved resolutions computed as ((localization precision)²+(Nyquist resolution)²)^{1/2} were quantified to compare the imaging speeds of DNA-PAINT and FRET-PAINT. The localization precisions of DNA-PAINT and FRET-PAINT were 6.9 nm and 11.1 nm, respectively.

The results are shown in FIGS. 2a to 2e.

Since 18000 frames in total were recorded at a frame rate of 10 Hz for DNA-PAINT, the total imaging time was 30 min (FIG. 2a). On the other hand, since 600 frames in total were recorded at a frame rate of 10 Hz for FRET-PAINT, the total imaging time was 60 s (FIG. 2b). The imaging speed of FRET-PAINT increased 29-fold compared to DNA-PAINT (FIG. 2c). The average values measured for the different areas revealed a 32-fold increase of the imaging speed on average (FIG. 2d). The imaging speeds of DNA-PAINT and FRET-PAINT were compared based on their convolved resolutions, and as a result, a 36-fold increase was obtained in the imaging speed of FRET-PAINT (FIG. 2e).

### Example 3: FRET-PAINT Multiplexed Imaging

The multiplexing capability of FRET-PAINT microscopy was assessed by the following experimental procedure.

Microtubules and mitochondria of COS-7 cells were immunostained using anti-tubulin antibody and anti-Tom20 antibody, respectively. The anti-tubulin antibody and anti-Tom20 antibody were orthogonally conjugated with Docking_P1 and Docking_P2, respectively. In the present invention, two approaches were used for multiplexed imaging.

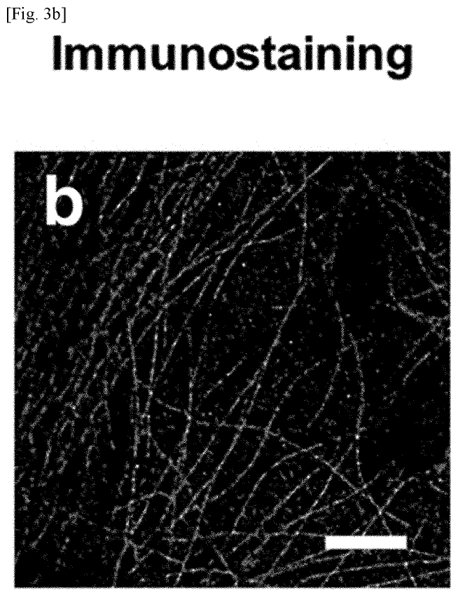

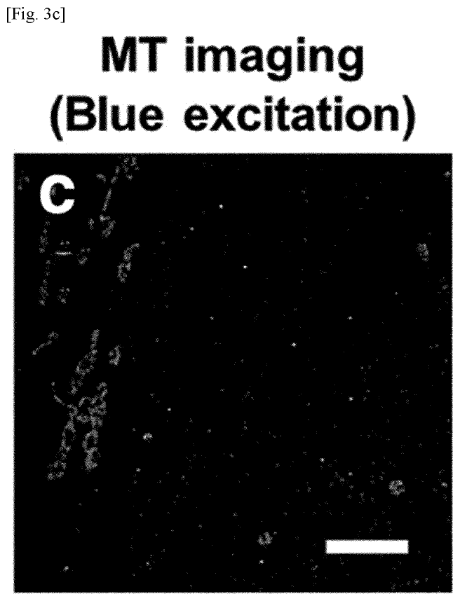

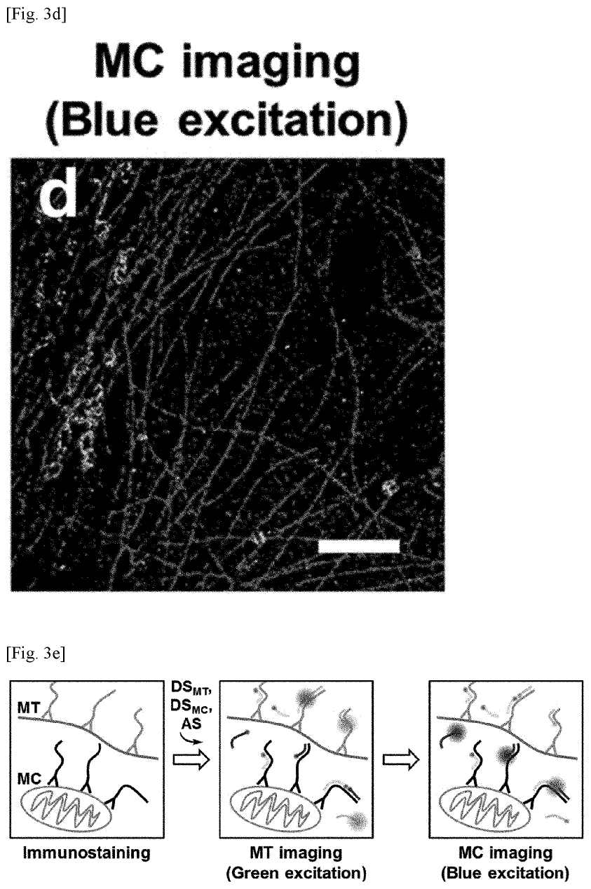

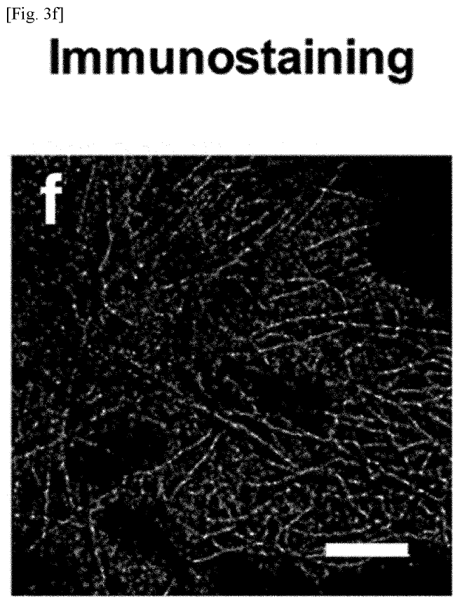

The results are shown in FIGS. 3a to 3h. In the one approach shown in FIG. 3a (by sequential treatment with donor and acceptor strands), microtubules were first imaged by injecting 20 nM Donor_P1_Alexa488 and 10 nM Acceptor_P2_Cy5 (FIG. 3b) and mitochondria were then imaged by injecting 10 nM Donor_P2_Alexa488 and 10 nM Acceptor_P2_Cy5 (FIGS. 3c and 3d). In the second approach shown in FIG. 3e (by simultaneous treatment with donor and acceptor strands), after injection of all DNA probes (10 nM Donor_P1_Cy3 for microtubules, 20 nM Donor_P2_Alexa488 for mitochondria, and 10 nM Acceptor_P2′_Cy5 for both microtubules and mitochondria) at the same time, microtubules were first imaged with Cy3 excitation (FIG. 3f) and mitochondria were then imaged with Alexa488 excitation (FIGS. 3g and 3h).

Even though there was no difference in imaging time between the two approaches, a disadvantage of the second approach (by simultaneous treatment with donor and acceptor strands) is a cross-talk between microtubule and mitochondria images. Thus, it was demonstrated that the sequential treatment with donor and acceptor strands is a better way to do multiplexing imaging.

### Example 4: Measurement of Biomarkers in a Broad Concentration Range

The ability to measure biomarkers in a broad concentration range was assessed by the following experimental procedure.

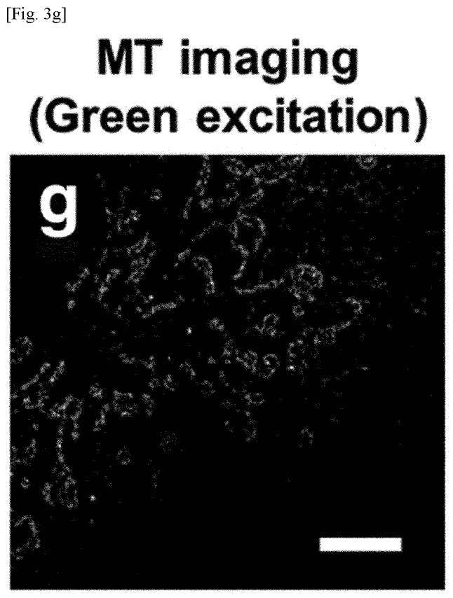

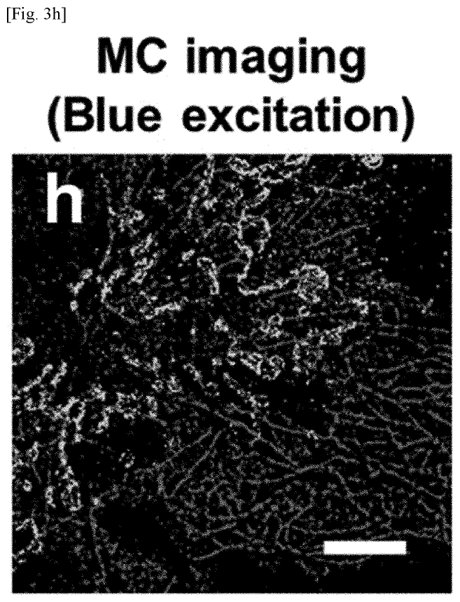

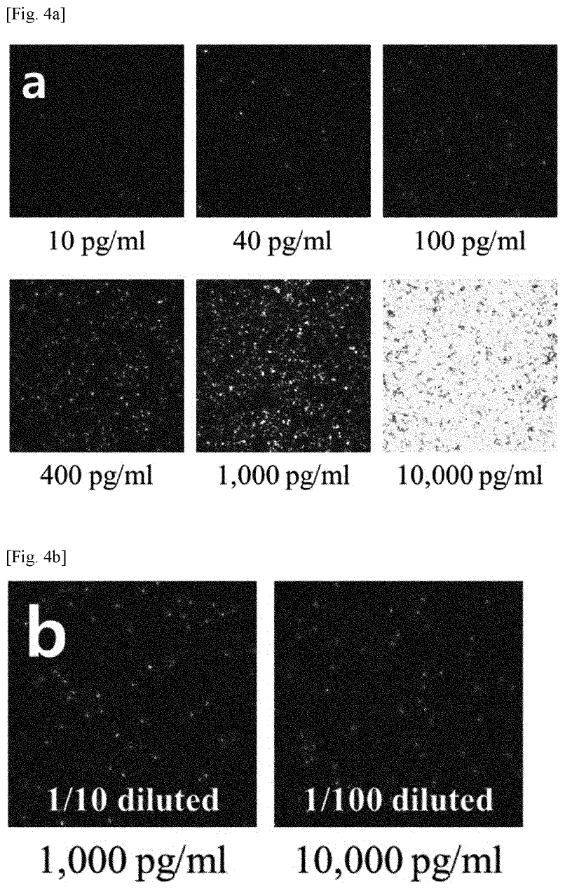

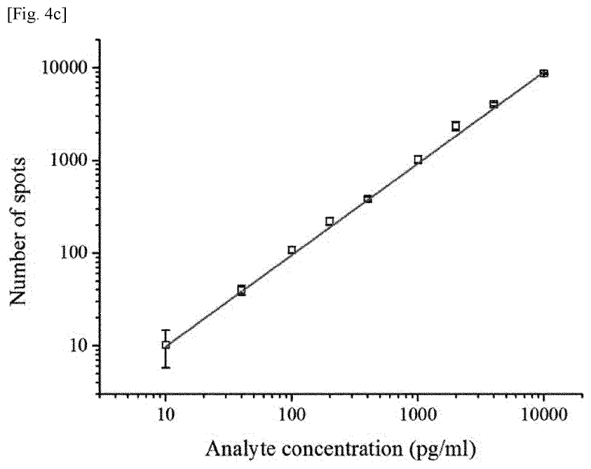

Biomarkers at concentrations of 10, 40, 100, 400, 1000, and 10000 pg/ml were immobilized in different chambers and the numbers of spots (individual biomarker molecules) in images were measured using donor and acceptor strands (FIG. 4a). The number of spots in images increased in proportion to the concentration. The spots started to overlap at concentrations of >1000 pg/ml, making it impossible to accurately count their number.

Biomarkers at concentrations of 1000 and 10000 pg/ml were 1/10 and 1/100 diluted and immobilized in different chambers and the numbers of spots in images were measured using donor and acceptor strands (FIG. 4b).

The numbers of spots counted at the indicated concentrations are plotted (FIG. 4c). The 1000 pg/ml data were obtained by counting the number of spots after 1/10 dilution and multiplying the number by 10 and the 10000 pg/ml data were obtained by counting the number of spots after 1/100 dilution and multiplying the number by 100. When the data were fitted to linear functions, the correlation coefficient was calculated to be 0.99997. From this, it was concluded that accurate measurement results can be obtained even after biomarkers at high concentrations are diluted.

### INDUSTRIAL APPLICABILITY

As discussed above, the method of the present invention enables the detection of various biomarkers from a very small amount of biofluid with a 1000-fold higher sensitivity than conventional methods. Therefore, the method of the present invention is useful for the early diagnosis of various diseases, including cancer, while solving the problems of conventional diagnostic methods using biomarkers.

1. A method for multiplexed detection of biomarkers, comprising a) attaching one or more types of biomarkers present in a sample taken from a subject to the surface of a substrate, b) allowing docking strand-conjugated detection antibodies to specifically bind to the biomarkers, c) binding imager strands labeled with fluorescent molecules or combinations of donor and acceptor strands to the docking strands to generate a fluorescence signal, and d) detecting the fluorescence signal, wherein steps c) and d) are repeated as many times as the number of the biomarker types by removing the used imager strands or the used donor and acceptor strands and introducing new imager strands or new combinations of donor and acceptor strands to the docking strands.

2. The method according to claim 1, wherein the imager strands have different sequences depending on the biomarker types.

3. The method according to claim 1, wherein the donor strands and the acceptor strands have different sequences depending on the biomarker types.

4. The method according to claim 1, wherein the sequences of the donor strands are different from those of the acceptor strands and are different from each other depending on the biomarker types; and the acceptor strands have the same sequence.

5. The method according to claim 1, wherein the sequences of the acceptor strands are different from those of the donor strands and are different from each other depending on the biomarker types; and the donor strands have the same sequence.

6. The method according to claim 1, wherein the docking strands comprise sequences complementary to the sequences of the imager strands or sequences complementary to the sequences of both the donor strands and the acceptor strands.

7. The method according to claim 1, wherein the sample is selected from the group consisting of blood, plasma, serum, saliva, tissue fluid, and urine.

8. The method according to claim 1, wherein the sample is diluted with a biomarker-free solution.

9. The method according to claim 1, wherein the same sample is diluted in different ratios.

10. The method according to claim 1, wherein the detection antibodies are selected from the group consisting of scFv, Fab, nanobodies, and immunoglobulin molecules.

11. The method according to claim 1, wherein the detection antibodies are primary antibodies and the docking strands are conjugated to the primary antibodies.

12. The method according to claim 1, wherein the detection antibodies are a combination of primary antibodies and secondary antibodies and the docking strands are conjugated to the secondary antibodies.

13. The method according to claim 1, wherein the fluorescent molecules are selected from the group consisting of Alexa Fluor, Atto, BODIPY, CF, Cy, and DyLight Fluor.

14. The method according to claim 1, wherein the peak wavelength of the emission spectrum of the fluorescent molecules labeled on the donor strands is shorter than the peak wavelength of the absorption spectrum of the fluorescent molecules labeled on the acceptor strands.

15. The method according to claim 1, wherein the fluorescence signal is generated when the imager strands bind to the docking strands or combinations of the donor and acceptor strands bind to the docking strands.

16. The method according to claim 1, wherein the biomarkers are proteins or metabolites.

17. A kit for multiplexed detection of biomarkers, comprising a) one or more types of detection antibodies conjugated with docking strands and b) i) one or more types of imager strands labeled with fluorescent molecules or ii) one or more types of donor strands labeled with fluorescent molecules and one or more types of acceptor strands labeled with fluorescent molecules wherein the kit detects a signal generated when the detection antibodies conjugated with the docking strands bind to biomarkers and the imager strands or the donor and acceptor strands bind to the docking strands.

18. The kit according to claim 17, wherein the kit comprises i) a substrate, ii) capture antibodies, iii) primary detection antibodies conjugated with docking strands, or primary detection antibodies unconjugated with docking strands and secondary detection antibodies conjugated with docking strands, and iv) fluorescent molecule-labeled imager strand, or donor and acceptor strands.

19. The kit according to claim 17, wherein the docking strands, the imager strands, the donor strands, and the acceptor strands are individually selected from the group consisting of nucleic acids and nucleic acid analogs.

20. The kit according to claim 18, wherein the substrate surface is directly attached with biomarkers present in a sample or is modified such that the capture antibodies capable of capturing biomarkers are attached to the substrate surface.

21. The kit according to claim 18, wherein the capture antibodies are attached to the substrate surface.

22. The kit according to claim 18, wherein the substrate is selected from the group consisting of slide glass, coverslips, quartz, and plastics.

23. A method for multiplexed detection of biomarkers, comprising a) attaching one or more types of RNAs present in a sample taken from a subject to the surface of a substrate, b) binding imager strands labeled with fluorescent molecules or combinations of donor and acceptor strands to the RNAs to generate a fluorescence signal, and c) detecting the fluorescence signal, wherein steps b) and c) are repeated as many times as the number of the RNA types by removing the used imager strands or the used donor and acceptor strands and introducing new imager strands or different combinations of donor and acceptor strands to the RNAs.

24. The method according to claim 23, wherein the sample is diluted with an RNA-free solution.

25. The method according to claim 23, wherein the same sample is diluted in different ratios.

26. A kit for multiplexed detection of biomarkers, comprising a) i) one or more types of imager strands labeled with fluorescent molecules or ii) one or more types of donor strands labeled with fluorescent molecules and one or more types of acceptor strands labeled with fluorescent molecules wherein the kit detects a signal generated when the imager strands or the donor and acceptor strands bind to RNAs.

27. The kit according to claim 26, wherein the kit comprises i) a substrate, ii) capture probes, iii) fluorescent molecule-labeled imager strands, or donor strands, and acceptor strands.

28. The kit according to claim 27, wherein the capture probes, the imager strands, the donor strands, and the acceptor strands are individually selected from the group consisting of nucleic acids and nucleic acid analogs.

29. The kit according to claim 27, wherein the substrate is attached with the capture probes or is surface-modified such that the capture probes can be attached to the substrate surface.

30. The kit according to claim 27, wherein the substrate is selected from the group consisting of slide glass, coverslips, quartz, and plastics.


SEQUENCE LISTING (3 sequences)

SEQ ID NO: 1 - DNA, 22 bp, Artificial Sequence
  Synthezied
ttgatctacatattcttcatta

SEQ ID NO: 2 - DNA, 9 bp, Artificial Sequence
  Synthesized
taatgaaga

SEQ ID NO: 3 - DNA, 10 bp, Artificial Sequence
  Synthesized
tatgtagatc